(19) United States
(12) Patent Application Publication
(10) Pub. No.: US 2010/0318014 A1
(43) Pub. Date: Dec. 16, 2010

(54) TREATMENT OF ACUTE ISCHEMIC BRAIN STROKE WITH OZONE

(76) Inventors: Joseph S. Latino, Brooklyn, NY (US); Steven A. Keyser, Salt Lake City, UT (US)

Correspondence Address: MORRISS OBRYANT COMPAGNI, P.C., 734 EAST 200 SOUTH, SALT LAKE CITY, UT, 84102, US

(21) Appl. No.: 12/819,871

(22) Filed: Jun. 21, 2010

Related U.S. Application Data
(63) Continuation-in-part of application No. 12/813,371, filed on Jun. 10, 2010.
Application No. 12/813,371 is a division of application No. 10/963,477, filed on Oct. 11, 2004, now Pat. No. 7,736,494.
Application No. 10/963,477 is a continuation-in-part of application No. 10/910,485, filed on Aug. 2, 2004, now abandoned.
(60) Provisional application No. 61/269,087, filed on Jun. 19, 2009.
(60) Provisional application No. 60/553,774, filed on Mar. 17, 2004.
(60) Provisional application No. 60/491,997, filed on Jul. 31, 2003.

(51) Int. Cl. A61M 1/36; A61M 37/00

(52) U.S. Cl. 604/6.14; 604/24

(57) ABSTRACT

Methods are provided for treatment of acute ischemic brain stroke based on the delivery of a measured amount of ozone to a sample of a mammalian patient's blood, blood fractionate or other biological fluid through the use of an ozone delivery system. The ozone-treated fluid, having absorbed a quantifiable absorbed-dose of ozone, is subsequently reinfused into the same patient and the autologous blood sample provides therapeutic effects to the patient, such as reduction in edema associated with the ischemic penumbra, improvement in impaired blood flow to the area surrounding the infarct, relaxation of the vascular endothelium and reduction of inflammation.

### CROSS REFERENCE TO RELATED APPLICATIONS

This is a non-provisional application claiming priority to provisional Ser. No. 61/269,087, filed Jun. 19, 2009, and this non-provisional application also claims priority to co-pending Ser. No. 12/813,371, filed Jun. 10, 2010, which is a divisional application of Ser. No. 10/963,477, filed Oct. 11, 2004, which is a continuation-in-part of Ser. No. 10/910,485, filed Aug. 2, 2004, which claims priority to both provisional application Ser. No. 60/553,774, filed Mar. 17, 2004, and provisional application Ser. No. 60/491,997, filed Jul. 31, 2003. The contents of all foregoing applications are incorporated herein, in their entirety, by reference.

### BACKGROUND OF THE INVENTION

1. Field of the Invention

This invention relates to therapeutic treatments for ischemic brain stroke and its attendant conditions, and specifically relates to therapeutic treatments of ischemic brain stroke conditions based treating fluids with a quantifiable absorbed dose of ozone via an ozone delivery system.

2. Statement of the Relevant Art

The references discussed herein are provided solely for the purpose of describing the field relating to the invention. Nothing herein is to be construed as an admission that the inventors are not entitled to antedate a disclosure by virtue of prior invention. Furthermore, citation of any document herein is not an admission that the document is prior art, or considered material to patentability of any claim herein, and any statement regarding the content or date of any document is based on the information available to the applicant at the time of filing and does not constitute an affirmation or admission that the statement is correct.

Stroke is the second leading cause of death worldwide and is responsible for 4.4 million (9 percent) of the total 50.5 million deaths each year. In the United States, stroke is the No. 3 cause of death behind heart disease, to which it is closely linked, and cancer. It affects more than 800,000 annually in the U.S., of which 600,000 are initial attacks and 200,000 are recurrent. At current trends, this number is projected to jump to one million annually by the year 2050. Stroke represents the leading cause of disability in the U.S. with more than 4 million people living with the after-effects of an attack.

Stroke is characterized by the sudden loss of circulation to an area of the brain, resulting in a corresponding loss of neurologic function. Also called cerebrovascular accident or stroke syndrome, stroke is a nonspecific term encompassing a heterogeneous group of pathophysiologic causes, including thrombosis, embolism and hemorrhage. Strokes currently are classified as either hemorrhagic or ischemic. Acute ischemic stroke refers to strokes caused by thrombosis or embolism, and accounts for approximately 87% of all strokes.

Ischemic stroke is caused by a blockage in a blood vessel that stops the flow of blood and deprives the surrounding brain tissue of oxygen. In the absence of oxygen, the brain cells in the immediate area begin to die and release a cascade of toxic chemicals that threaten brain tissue in the surrounding area. This phenomenon is referred to as the ischemic penumbra.

Current statistics indicate that 7.6 percent of individuals that suffer an ischemic stroke die within 30 days of the episode. Moreover, approximately 25 percent of all individuals die within a year of their first stroke. Fourteen percent of stroke patients will suffer a stroke relapse within one year, and within five years, the rate of relapse escalates to 25 percent. Fifty percent of stroke victims that survive experience moderate to severe impairment requiring special care including nursing home care or other long-term care facility treatment. When the direct costs (care and treatment) and indirect costs (lost productivity) of strokes are considered together, the total cost of stroke to the United States is estimated in 2008 at $65 billion per year, 87% of which is attributed to ischemic stroke.

Pathophysioloqy of Stroke: On the macroscopic level, ischemic strokes most often are caused by extracranial embolism or intracranial thrombosis. On the cellular level, any process that disrupts blood flow to a portion of the brain unleashes an ischemic cascade, leading to the death of neurons and cerebral infarction.

Thrombotic stroke is caused by a thrombus (blood clot) that develops in an artery supplying blood to the brain. This is usually because of a repeated buildup of fatty deposits, calcium and clotting factors, such as fibrinogen and cholesterol, carried in the blood. The body perceives the buildup as an injury to the vessel wall and responds by forming blood clots. These blood clots get trapped onto the plaque on the vessel walls, eventually stopping blood flow.

There are two types of thrombotic stroke, based upon vessel diameter. Large vessel thrombosis, the most common form of thrombotic stroke, occurs in the brain's larger arteries. The impact and damage tends to be magnified because all the smaller vessels that the artery feeds are deprived of blood. In most cases, large vessel thrombosis is caused by a combination of long-term plaque buildup (atherosclerosis) followed by rapid blood clot formation. Small vessel disease, also referred to as lacunar infarction, occurs when blood flow is blocked to a very small arterial vessel. It has been linked to high blood pressure (hypertension) and is an indicator of atherosclerotic disease. Thrombotic disease accounts for about 60 percent of acute ischemic strokes. Of those, approximately 70 percent are large vessel thrombosis.

In embolic stroke, a clot forms outside of the brain, usually in the heart or large arteries of the upper chest and neck, and is transported through the bloodstream to the brain. There, it eventually reaches a blood vessel small enough to block its passage. Emboli can be fat globules, air bubbles or, most commonly, pieces of an atherosclerotic plaque (i.e. lipid debris) that have detached from an artery wall. Many emboli are caused by a cardiac condition called atrial fibrillation causing blood to pool and form clots that can travel to the brain and cause a stroke. Cardiac sources of embolism account for 80 percent of embolic ischemic strokes.

Within seconds to minutes of the loss of perfusion to a portion of the brain, an ischemic cascade is initiated. Allowed to progress, it will cause a central area of irreversible infarction surrounded by an area of potentially reversible ischemic penumbra.

On the cellular level, the ischemic neuron becomes depolarized as ATP is depleted and membrane ion-transport systems fail. The resulting influx of calcium leads to the release of a number of neurotransmitters, including large quantities of glutamate, which in turn activates N-methyl-D-aspartate (NMDA) and other excitatory receptors on other neurons. These neurons then become depolarized, causing further calcium influx, further glutamate release, and local amplification of the initial ischemic insult. This massive calcium influx also activates various degradative enzymes, leading to the destruction of the cell membrane and other essential neuronal structures. This metabolic aberration creates an intracellular gradient responsible for intracellular accumulation of water (cytotoxic edema).

Within hours to days after a stroke, specific genes are activated, leading to the formation of cytokines and other factors that, in turn, cause further inflammation and microcirculatory compromise. Cerebral endothelial cells are more resistant to ischemia than are neuronal cells. About 2-4 hours after the onset of ischemia, the integrity of the blood-brain barrier becomes compromised, and plasma proteins are able to pass into the extracellular space. The intravascular water follows when reperfusion occurs (vasogenic edema). This process reportedly begins 6 hours after the onset of stroke and reaches a maximum 2-4 days after the onset of stroke. Reperfusion can also be accompanied by hemorrhagic transformation of the infarct, which is usually related to the volume and site of the infarct.

This vascular inflammation may be due to an imbalance between pro-inflammatory (e.g. interferon gamma, TNF-gamma, IL-6 and IL-12) and anti-inflammatory (e.g. interleukin-4 and IL-10) cytokines release by immune-modulatory T cells associated within the infarct site and ischemic penumbra. In addition, there is evidence indicating that the vascular endothelium plays a major role in the regulation of blood flow and is of importance in connection with cardiovascular disorders, including acute ischemic brain stroke. Dysfunctional endothelium has been suggested as a contributory factor in many ischemic disorders and may play a role in the demise of the ischemic penumbra. Ultimately, the ischemic penumbra is consumed by these progressive insults, coalescing with the infarcted core, often within hours of the onset of the stroke.

The central goal of therapy in acute ischemic stroke is to preserve the ischemic penumbra. This can be accomplished by limiting the severity of ischemic injury (i.e. neuronal protection) or reducing the duration of ischemia (i.e. restoring blood flow to the compromised area). The timing of restoring cerebral blood flow appears to be a critical factor in the preservation of the ischemic penumbra and may prove pivotal in neuronal protection as well.

### Apoptosis

Apoptosis specifically refers to an energy-dependent, asynchronous, genetically controlled process by which unnecessary or damaged single cells self-destruct when apoptosis genes are activated (Martin, S J 1993; Earnshaw, W C 1995). There are three distinct phases of apoptosis. Initially, the cell shrinks and detaches from neighboring cells. The nucleus is broken down with changes in DNA including strand breakage (karyorhexis) and condensation of nuclear chromatin (pyknosis). In the second phase, nuclear fragments and organelles condense and are ultimately packaged in membrane-bound vesicles, exocytosed and ingested by surrounding cells. In final phase, membrane integrity is finally lost and permeability to dyes (i.e. trypan blue) occurs. The absence of inflammation differentiates apoptosis from necrosis when phagocytized by macrophages and epithelial cells (Kam, P C A 2000).

In contrast, necrotic cell death is a pathological process caused by overwhelming noxious stimuli (Lennon, S V 1991). Synchronously occurring in multiple cells, it is characterized by cell swelling or “oncosis,” resulting in cytoplasmic and nuclear swelling and an early loss of membrane integrity. Bleb formation (blister-like, fluid filled structures) of the plasma membrane occurs, in which ultimate rupture may occur causing an influx of neutrophils and macrophages in the surrounding tissue, and leading to generalized inflammation (Majno, G 1995).

Apoptotic Inducers: Four main groups of stimuli for apoptosis have been reported: ionizing radiation and alkylating anticancer drugs causing DNA damage, receptor mechanism modulation (i.e. glucocorticoids, tumor necrosis factor-α, nerve growth factor or interleukin-3), enhancers of apoptotic pathways (i.e. phosphatases and kinase inhibitors), and agents that cause direct cell membrane damage and include heat, ultraviolet light and oxidizing agents (i.e. superoxide anions, hydroxyl radicals and hydrogen peroxide) (Kam, P C A 2000).

In addition to the oxidizing agents, many chemical and physical treatments capable of inducing apoptosis are also known to evoke oxidative stress (Buttke, M 1994, Chandra, J 2000). Ionizing and ultraviolet radiation both generate reactive oxygen intermediates such as hydrogen peroxide and hydroxyl free radicals. Low doses of hydrogen peroxide (10-100 μM) induce apoptosis in a number of cell types directly establishing oxidative stress as a mediator of apoptosis. However, high doses of this oxidant induce necrosis, consistent with the concept that the severity of the insult determines the form of cell death (apoptosis vs. necrosis) that occurs. A free radical is not a prerequisite for inducing apoptosis; doxorubucin, cisplatin and ether-linked lipids are anti-neoplastics that induce apoptosis and oxidative damage.

Alternatively, oxidative stress can be induced by decreasing the ability of a cell to scavenge or quench reactive oxygen intermediates (ROI) (Buttke, M 1994). Drugs (i.e. butathionine sulfoxamine) that reduce intracellular glutathione (GSH) render cells more susceptible to oxidative stress-induced apoptosis. Cell studies report a direct relationship between extracellular catalase levels and sensitivity to hydrogen peroxide-induced apoptosis. Apoptosis induced through tumor necrosis factor-α stimulation has been demonstrated to be associated with an increase in intracellular ROI. However, this apoptosis has been inhibited by the addition of a number of antioxidants; thioredoxin, a free radical scavenger, and N-acetylcysteine, an antioxidant and GSH precursor.

Modulation of inflammatory cytokine production by apoptotic cells: There is growing evidence that apoptotic neutrophils have an active role to play in the regulation and resolution of inflammation following phagocytosis by macrophages and dendritic cells. A hallmark of phagocytic removal of necrotic neutrophils by macrophages is an inflammatory response including the release of proinflammatory cytokines (Vignola, A M 1998, Beutler, B 1988, Moss, S T 2000, Fadok V A, 2001). In contrast, apoptotic neutrophil clearance is not accompanied by an inflammatory response; phagocytosis of these apoptotic cells has been shown to inhibit macrophage production of pro-inflammatory cytokines (GM-CSF, IL-1β, IL-8, TNF-α, TxB2, and LTC4) with a concomitant activation of anti-inflammatory cytokine production (TGF-β1, PGE2 and PAF) (Fadok, V A 1988, Cvetanovic, M 2004), This phenomenon of suppression of proinflammatory cytokine production by macrophages has been extended to include phagocytosis of apoptotic lymphocytes (Fadok, V A 2001).

In addition to macrophages, down regulation of proinflammatory cytokine release in response to apoptotic cells has also been demonstrated by non-phagocytizing cells including human fibroblasts, smooth muscle, vascular endothelial, neuronal and mammary epithelial cells (Fadok, V A 1988, 2000; McDonald, P P 1999, Cvetanovic M, 2006). Apoptotic neutrophils in contact with activated monocytes elicit an immunosuppressive cytokine response, with enhanced IL-10 and TGF-β production and only minimal TNF-α and IL-1β cytokine production (Byrne, A 2002). Byrne et al. concluded that the interaction between activated monocytes and apoptotic neutrophils may create a unique response, which changes an activated monocyte from being a promoter of the inflammatory cascade into a cell primed to deactivate itself and other cellular targets.

Techniques to Identify Apoptosis: Techniques to identify and quantify apoptosis, and distinguish this event from necrosis, may include staining with nuclear stains allowing visualization of nuclear chromatin clumping (i.e. Hoeschst 33258 and acridine orange) (Earnshaw, W C 1995). Accurate identification of apoptosis is achieved with methods that specifically target the characteristic DNA cleavages. Agarose gel electrophoresis of extracted DNA fragments yields a characteristic ‘ladder’ pattern which can be used as a marker for apoptosis (Bortner, C D 1995). A lesser extent of DNA degradation produces hexameric structures called ‘rosettes’ where necrotic cells leave a nondescript smear (Pritchard, D M 1996). Terminal transferase deoxyuridine nick-end labeling of DNA break points (TUNEL method), which labels uridine residues of the nuclear DNA fragments, can also be used to quantify apoptosis (Gavrieli, Y 1992).

Several signature events in the process of apoptosis may also be quantified by flow cytometry. These include dissipation of the mitochondrial membrane potential which is an early apoptotic event, externalization of phosphotidylserine through capture with annexin V, loss of plasma membrane integrity and nuclear chromatin condensation (distinguishing live, apoptotic and necrotic cells), and activation of caspase enzymes (early stage feature of apoptosis) (Technical Bulletin—InVitrogen 2004).

Release of Vasorelaxatory Agents Induced by Oxidative Stress: Vascular endothelial cells, including human umbilical vein endothelial cells (HUVECs), are known to release potent vasodilators, including nitric oxide (NO) and prostacyclins. Treatment of HUVECs with ozonated serum, an oxidative stressor, results in a significant and steady increase in NO production. Moreover, during twenty-four (24) hour HUVEC incubation with ozonated serum, inhibition of E-selectin release (a proinflammatory mediator) and no effect on endothelin-1 production (a potent vasoconstrictor) has been reported (Valacchi, G 2000). Valacchi et al. has suggested that reinfusion of ozonated blood into patients, by enhancing release of NO, may induce vasodilation in ischemic areas and reduce hypoxia.

### C-Reactive Protein (CRP)

CRP is a product of inflammation the synthesis of which by the liver is stimulated by cytokines in response to an inflammatory stimulus. CRP activates the classic complement pathway and participates in the opsonization of ligands for phagocytosis. Initially suggested as solely a biomarker and powerful predictor of cardiovascular risk, CRP now appears to be a mediator of atherogenesis. CRP has a direct effect on promoting atherosclerotic processes and endothelial cell activation. CRP potently down regulates endothelial nitric oxide synthase (eNOS) transcription and destabilizes eNOS mRNA, which decreases both basal and stimulated nitric oxide (NO) release.

In a synchronous fashion, CRP has been shown to stimulate endothelin-1 (potent vasoconstrictor) and interleukin-6 release (pro-inflammatory cytokine), upregulate adhesion molecules, and stimulate monocyte chemotactic protein-1 while facilitating macrophage LDL uptake. More recently, CRP has been shown to facilitate endothelial cell apoptosis and inhibit angiogenesis, as well as potentially upregulate nuclear factor kappa-B, a key nuclear factor that facilitates the transcription of numerous pro-atherosclerotic genes.

The direct pro-atherogenic effects of CRP extend beyond the endothelium to the vascular smooth muscle where it directly upregulates angiotensin type 1 receptors and stimulates vascular smooth muscle migration, proliferation, neointimal formation and reactive oxygen species production. CRP has several deleterious effects (e.g., reduced survival, differentiation, function, apoptosis, and endothelial progenitor cell-eNOS mRNA expression) on endothelial progenitor cells which are important in neovascularization including induction of blood flow recovery in ischemic limbs and increase in myocardial viability after infarction.

A variety of imaging techniques are available to assess the degree of edema surrounding the infarct site and blood flow to the ischemic penumbra in ischemic brain stroke patients. Examples of such imaging techniques are discussed below.

Head Computerized Axial Tomography (CT scan): Emergent non-contrast head CT scanning is mandatory for rapidly distinguishing ischemic from hemorrhagic infarction and for defining the anatomic distribution of stroke. Most patients who have had onset of ischemic stroke symptoms within 6 hours initially will have normal findings on CT scan. After 6-12 hours, sufficient edema is recruited into the stroke area to produce a regional hypodensity on CT scan. A large hypodense area present on CT scan within the first 3 hours of symptom onset should prompt careful re-questioning regarding the time of stroke symptom onset.

Transcranial Doppler (TCD): In TCD, a probe is placed over areas on the head to detect blood velocity and pressure in certain arteries at various depths in the brain. In the early hours after occlusive stroke, TCD allows the assessment of the location and extent of occlusions or atheromatous plaques in extracranial carotid and large intracranial vessels, including the middle cerebral and basilar arteries.

Magnetic Resonance Imaging: Despite initial screening by CT to distinguish ischemic from hemorrhagic stroke, MRI has demonstrated greater accuracy in the identification of the acute infarction and greater predictive accuracy in the degree of lesion volume of the ischemic penumbra. One method of MRI analysis, diffusion weighted imaging (DWI), reflects the microscopic random motion of water molecules and is highly sensitive to early changes immediately following stroke onset.

For example, in the hyperacute phase of the ischemic stroke (0-24 hr), MRI is able to detect ischemic changes within minutes of onset. A few hours after stroke onset, MRI analysis can detect early signature events ascribed to cytotoxic edema. After 8 hours, MRI signals are interpreted as to discern changes associated with cytotoxic and vasogenic edema. Enhanced sensitivity to subtle changes in the acute (1-7 days), subacute (7-21 days) and chronic phases (>21 days) of the ischemic stroke process by MRI has led to its increased use in the diagnosis and management of acute ischemic stroke.

Perfusion-weighted imaging (PWI) is an MRI technique that yields information about the perfusion status of the brain. It can be used to estimate cerebral blood volume. Coupled with arterial input the relative cerebral blood flow can be calculated. DWI and PWI together have been shown to be highly sensitive to the early phases (up to 48 hours) after the onset of stroke. In conjunction, they provide information about location and extent of infarction within minutes of onset; when performed in series, they can provide information about the pattern of evolution of the ischemic lesion.

The physical symptoms of an acute ischemic stroke provide objective evidence of brain ischemia, including the initial infarction and resulting edema, due to an obstructed or reduced blood flow. Contributory factors may include vascular inflammation and a dysfunctional endothelium. Some of the more common symptoms of stroke include loss of (or abnormal) sensations in an arm, leg or one side of the body, weakness or paralysis of an arm or leg or one side of the body (including asymmetrical facial expressions—facial palsy), partial loss of vision [gaze paresis (slight or partial paralysis) or hemianopia (blindness in one half of the visual field of one or both eyes)] and hearing. Additional physical symptoms include, double vision (diplopia), dizziness (including syncope), slurred speech (dysarthria—Difficulty in articulating words, caused by impairment of the muscles used in speech), problems thinking of or saying the right word (aphasia—partial or total loss of the ability to articulate ideas or comprehend spoken or written language), inability to recognize parts of the body (hemi-inattention, extinction or anosognosia—failure to recognize paralysis), and imbalance and falling (including limb ataxia—loss of the ability to coordinate muscular movement).

There are a number of standard instruments that have been designed for patient assessment in stroke. Outcome measures are typically selected on the basis of their reliability, familiarity to the neurologic community, and adaptability for use in patients who have had a stroke. Examples of four stroke assessment tools that meet these criteria include the following described methods.

The Barthel index (measures of disability/activities of daily living) is a reliable and valid measure of the ability to perform activities of daily living such as eating, bathing, walking, and using the toilet. Patients able to perform all activities with complete independence are given a score of 100.

The modified Rankin scale (global disability scale) is a simplified overall assessment of function in which a score of 0 indicates the absence of symptoms and a score of 5, severe disability.

The Glasgow outcome scale (level-of-consciousness scale) is a global assessment of function in which a score of 1 indicates a good recovery; a score of 2, moderate disability; a score of 3, severe disability; a score of 4, survival but in a vegetative state; and a score of 5, death.

The National Institutes of Health Stroke Scale (NIHSS; stroke deficit scale), a serial measure of neurologic deficit, is a 42-point scale that quantifies neurologic deficits in 11 categories. For example, a mild facial paralysis is given a score of 1, and complete right hemiplegia with aphasia, gaze deviation, visual-field deficit, dysarthria, and sensory loss is given a score of 25. Normal function without neurologic deficit is scored as zero.

Other scales used to evaluate stroke patients may include: pre-hospital stroke assessment tools [i.e. Cincinnati Stroke Scale and Los Angeles Prehospital Stroke Screen (LAPSS)]; acute assessment scales [i.e. Canadian Neurological Scale (CNS), Glasgow Coma Scale (GCS), Hempispheric Stroke Scale, Hunt & Hess Scale, Mathew Stroke Scale, Mini-Mental State Examination (MMSE), Orgogozo Stroke Scale, Oxfordshire Community Stroke Project Classification (Bamford) and Scandinavian Stroke Scale]; functional assessment tools [i.e. Berg Balance Scale, Stroke Impact Scale (SIS), Stroke Specific Quality of Life Measure (SS-QOL)]; and, outcome assessment tools [i.e. American Heart Association Stroke Outcome Classification (AHA SOC), Functional Independence Measurement (FIM), and Health Survey SF-36 & SF-12].

There are a number of treatments that are currently used to ameliorate the effects of stroke, and to prevent future strokes, including the therapies discuss below.

Interventional Drug Therapy: Currently, tissue Plasminogen Activator (tPA) is the only thrombolytic agent (also known as a fibrinolytic or ‘clot-busting’ drug) approved by the Food and Drug Administration (FDA) for treating acute ischemic stroke. There are two ways to administer tPA, intravenously or intra-arterially directly to the clot site Despite an increased incidence of intracerebral hemorrhage, an improvement in clinical outcome at three months was found in patients treated with intravenous t-PA within three hours of the onset of acute ischemic stroke.

Aside from the severe restriction that an ischemic stroke patient can only receive tPA within a strict four and one half hour window from incident onset, patients receiving Vitamin-K antagonist therapy (i.e. warfarin), exhibit severely elevated blood pressure or blood sugar, exhibit a low platelet count, suffer from end-stage liver or kidney disorders, or have undergone recent surgery, are precluded from thrombolytic treatment. Currently, tPA therapy is appropriate for about 5 percent to 10 percent of stroke patients.

Attempts to widen the therapeutic window until six hours for tPA administration have evidenced no clear benefit of tPA therapy; a time period when a substantial number of patients present for evaluation. Therapeutic failure may have occurred because some patients treated 4.5 to 6 hours after symptom onset have already sustained severe, irreversible brain injury and others have already undergone spontaneous recanalization of their occluded arteries. Treatment of these patients is unlikely to produce beneficial effects and may result in harm secondary to brain hemorrhage.

Mechanical Clot Disruption: A majority of patients arrive at the hospital too late to qualify for intervention with tPA or have some other contraindications that effectively prohibit the use of the drug. An endovascular procedure involving the use of a cork-screw shaped device is the first FDA approved mechanical device for the treatment of ischemic stroke. This device is used on the end of a catheter to physically pull out all or part of a clot. The major limitation to the retriever device is that the clot must be visible and accessible in order for the physician to guide the catheter to the location of the clot. The Penumbra System™, comprising an aspiration platform containing multiple devices that are size-matched to the specific neurovascular anatomy allowing clots to be gently aspirated out of intracranial vessels, was approved by the FDA in 2008 for post stroke revascularization. Cerebral artery size, advanced surgical and imaging techniques, and vessel perforation significantly limit the adoption of these mechanical clot disruption technologies.

Drug Therapy: Administration of anticoagulants can play a role in preventing ischemic stroke and its recurrence. They are drugs used to prevent clot formation or to prevent a clot that has formed from enlarging. They cannot, however, dissolve clots that already have formed. Anticoagulant drugs fall into three categories: inhibitors of clotting factor synthesis (i.e. warfarin), inhibitors of thrombin, and antiplatelet drugs (i.e. aspirin, Clopidogrel, Eptifibatide, dipyridamole, and Ticlopidine).

In view of the limitations that are presented with currently practiced therapies, new approaches are being sought to reduce the frequency and severity of clinical sequelae secondary to acute ischemic brain stroke.

Historically, ozone has been used as a disinfectant or sterilizing agent in a wide variety of applications. These include fluid-based technologies such as: purification of potable water, sterilization of fluids in the semi-conductor industry, disinfection of wastewater and sewage, and inactivation of pathogens in biological fluids. Ozone has also been used in the past as a topical medicinal treatment, as a systemic therapeutic and as a treatment of various fluids that were subsequently used to treat a variety of diseases. Specifically, there have been numerous attempts utilizing a variety of ozone-based technologies to treat acute ischemic brain stroke in patients.

Previous technologies were incapable of measuring and differentiating between the amount of ozone that was delivered and the amount of ozone actually absorbed and used. This meant previous medicinal technologies used in patient treatment were incapable of measuring, reporting or differentiating the amount of ozone delivered from the amount of ozone that was actually absorbed and used. This problem made regulatory approval as a therapeutic unlikely.

In the treatment of acute ischemic brain stroke, previous technologies were also incapable of measuring, reporting or differentiating the amount of ozone delivered from the amount that was actually absorbed by the fluid and utilized by the patient. The inability to measure the amount of ozone absorbed may result in excessive absorption resulting in unacceptable levels of cellular necrosis in the leukocyte fraction of the treated blood, which, when reinfused, may result in promotion of an inflammatory response. Furthermore, any technology considered to treat acute ischemic brain stroke utilizing blood ex vivo with ozone may have to be able to maintain the biological integrity of the fluid for its subsequent intended therapeutic use.

In addition, early approaches of mixing ozone with fluids employed gas-fluid contacting devices that were engineered with poor mass transfer efficiency of gas to fluids. Later, more efficient gas-fluid contacting devices were developed, but these devices used construction materials that were not ozone inert and therefore, reacted with and absorbed ozone. This resulted in absorption of ozone by the construction materials making it impossible to determine the amount of ozone delivered to and absorbed by the fluid. Furthermore, ozone absorption by construction materials likely caused oxidation and the subsequent release of contaminants or deleterious byproducts of oxidation into the fluid.

Experimental research confirms the problem of ozone absorption by construction materials. An ozone/oxygen admixture at 1200 ppmv was passaged through a commercially available membrane oxygenator. For a period in excess of two hours, a majority of the ozone delivered to the device was absorbed by the construction materials. This data strongly suggests commercially-available membrane gas-fluid contacting devices, made from ozone reactive materials, cannot be used with ozone, and supports the necessity for developing novel ozone-inert gas-fluid contacting devices.

In addition, prior methods do not quantify the amount of ozone that does not react with the biological fluid. The inability to measure residual-ozone has led to inaccurate and imprecise determination of the amount of ozone actually absorbed and utilized by the fluid.

Prior technologies also include inefficient methods of mixing ozone with fluids, yielding irregular exposure. For example, relatively large amounts of ozone may be exposed to some of the fluid and less to other portions. The result of this inefficient mixing causes a wide variation in the amount of ozone exposed to the fluid. This wide variation in ozone exposure may cause diverse biochemical events, including unacceptable levels of cellular necrosis in various portions of the fluid leading to untoward and irreproducible results.

Prior techniques also failed to recognize that fluids of varying composition display different absorption phenomena. The range of values for extracellular antioxidants in blood, including ascorbic acid (0.4-1.5 mg/dL), uric acid (2.1-8.5 mg/dL), bilirubin (0-1.0 mg/dL) and Vitamin A (30-65 μg/dL) and other oxidizable substrates such as cholesterol (140-240 mg/dL), LDL-cholesterol (100-159 mg/dL), HDL-cholesterol (33-83 mg/dL) and triglycerides (45-200 mg/dL), may alter the amount of ozone necessary to be delivered to the fluid and subsequently absorbed and utilized to achieve a desired clinical effect.

In view of the difficulties encountered in prior techniques of treating ischemic brain stroke conditions as noted above, it would be advantageous to provide new methodologies of treatment that assure accurate and effective treatment regimens.

### BRIEF SUMMARY OF THE INVENTION

In accordance with the present invention, methods of treatment of acute ischemic brain stroke, and related conditions or symptoms, are provided which comprise subjecting an amount of blood, blood fractionate or other biological fluid, extracorporeally, to an amount of ozone delivered by an ozone delivery system to provide for the absorption of a quantifiable absorbed-dose of ozone, and re-infusing the treated fluid into the patient. The methods of the present invention are effective in reducing edema associated with the ischemic penumbra, increasing blood flow to the area surrounding the infarct, which may include ischemic tissue and the ischemic penumbra, relaxation of the vascular endothelium and reduction of inflammation, as well as other therapeutic effects.

The methods of the invention further include reinfusion of the treated fluid having the quantified absorbed dose of ozone into the mammalian subject to provide and elicit therapeutic effects which treat the disease, condition or symptoms of the disclosed diseases, as well as other diseases.

The methods of the present invention further provide for the manufacture of substances or compositions that are useful in the therapeutic treatment of acute ischemic brain stroke and related symptoms and conditions thereof. The methods of the present invention further provide for the use of such substances and compositions in the manufacture of medicaments or other administrable substances for the therapeutic treatment of acute ischemic brain stroke and symptoms or conditions thereof.

Treatment of patients who are or have experienced acute ischemic brain stroke produces other therapeutic effects, including measured patient improvement from or in paralysis, motor weakness, loss of sensation, ocular and auditory functions, stroke-free survival, severity of recurrent stroke, cognitive function, verbal communication, re-attainment of independence, and improvement in overall survival.

The present invention is directed to providing methods in treating blood with ozone extracorporeally to generate leukocyte apoptosis, without excessive necrosis, sufficient to reduce edema associated with the ischemic penumbra, increase blood flow to the area surrounding the infarct, which may include ischemic tissue, and the ischemic penumbra, promote relaxation of the vascular endothelium and reduce inflammation once the treated blood is reinfused.

The present invention is further directed to methods of treating blood with ozone extracorporeally which, once reinfused, causes reduction in CRP sufficient to elicit clinical benefit.

The present invention is further directed to providing methods for the treatment of blood, blood fractionate or other fluid, and the use of this treated blood, blood fractionate or other fluid in the treatment of acute ischemic brain stroke in a mammalian patient by administration to the patient of such treated blood, blood fractionate or other fluid.

The present invention further comprises extracorporeally subjecting an aliquot of a mammalian patient's blood, or the separated cellular fractions of the blood, or mixtures of the separated cells, including platelets, to a measured amount of ozone such that the fluid absorbs a quantifiable absorbed-dose of ozone. On re-introduction of this autologous aliquot to the patient's body, the blood, blood fractionate or other fluid with a quantifiable absorbed-dose of ozone may affect improvement of any condition caused by stroke.

The present invention is further directed to methods for treatment of acute ischemic brain stroke, including a methods of delivery of a measured amount of ozone and subsequent absorption of a quantifiable absorbed-dose of ozone by blood or derivatives thereof extracorporeally, to cause or promote sufficient leukocyte apoptosis necessary to elicit clinical benefit when reinfused autologously into a patient.

The present invention is further directed to methods that induce apoptosis in the leukocyte fraction of blood, or a blood derivative, which has absorbed a quantifiable absorbed-dose of ozone to reduce inflammation when reinfused autologously into a patient suffering from stroke.

The present invention is further directed to inducing apoptosis in the leukocyte fraction of blood or blood derivative which has absorbed a quantifiable absorbed-dose of ozone without causing excessive necrosis, to reduce inflammation when reinfused autologously into a patient suffering from stroke.

The present invention is further directed to treating acute ischemic stroke by using blood or a blood derivative which has absorbed a quantifiable absorbed-dose of ozone preventing excessive necrosis which may be pro-inflammatory when reinfused autologously into a patient.

The present invention is directed to providing methods of treatment which reduce inflammation in patients suffering from acute ischemic stroke by a method comprising connecting a subject to a device for withdrawing blood, withdrawing blood and delivering a measured amount of ozone to the blood under conditions which may induce sufficient leukocyte apoptosis without excessive necrosis, wherein the treated blood is subsequently re-infused into the subject.

The methods of the present invention induce sufficient leukocyte apoptosis without excessive necrosis, which may be evaluated by a number of diagnostic methods including light microscopy with nuclear stains, electrophoretic analysis of DNA fragmentation, TUNEL analysis and multiparameter flow cytometry.

The methods of the present invention induce apoptosis in the leukocyte fraction of blood, or a blood derivative which has absorbed a quantifiable absorbed-dose of ozone to reduce edema in the ischemic penumbra of stroke patients when reinfused autologously.

The present methods are directed to inducing apoptosis in the leukocyte fraction of blood or blood derivative which has absorbed a quantifiable absorbed-dose of ozone to reduce edema in the area surrounding the infarct which may include ischemic tissue and the ischemic penumbra of stroke patients when reinfused autologously.

The methods of the present invention are directed to reducing edema in the ischemic penumbra in patients suffering from acute ischemic stroke and/or reducing edema in the area surrounding the infarct, which may include ischemic tissue and the ischemic penumbra, in patients suffering from acute ischemic stroke, by a method comprising connecting a subject to a device for withdrawing blood, withdrawing blood and delivering a measured amount of ozone to the blood under conditions which may induce sufficient leukocyte apoptosis without excessive necrosis wherein the treated blood is subsequently re-infused into the subject.

The present invention is further directed to inducing apoptosis in the leukocyte fraction of blood or blood derivative which has absorbed a quantifiable absorbed-dose of ozone to improve blood flow to the area surrounding the infarct which may include ischemic tissue and the ischemic penumbra of stroke patients when reinfused autologously.

The present invention is further directed to inducing apoptosis in the leukocyte fraction of blood or blood derivative which has absorbed a quantifiable absorbed-dose of ozone without causing excessive necrosis, to improve blood flow to the area surrounding the infarct which may include ischemic tissue and the ischemic penumbra of stroke patients when reinfused autologously.

The methods of the present invention are further directed to improving blood flow to the area surrounding the infarct, which may include ischemic tissue and the ischemic penumbra, in patients suffering from acute ischemic stroke, by a method comprising connecting a subject to a device for withdrawing blood, withdrawing blood and delivering a measured amount of ozone to the blood under conditions which may induce sufficient leukocyte apoptosis without excessive necrosis wherein the treated blood is subsequently re-infused into the subject.

The methods of the present invention are directed to inducing apoptosis in the leukocyte fraction of blood or blood derivative which has absorbed a quantifiable absorbed-dose of ozone without causing excessive necrosis, to relax the vascular endothelium of stroke patients when reinfused autologously.

The methods of the present invention are directed to relaxing the vascular endothelium in patients suffering from acute ischemic stroke, and/or reducing the edema in the ischemic penumbra of acute ischemic brain stroke patients, and/or reducing the edema in the area surrounding the infarct which may include ischemic tissue and the ischemic penumbra of acute ischemic brain stroke patients, by a method comprising connecting a subject to a device for withdrawing blood, withdrawing blood and delivering a measured amount of ozone to the blood under conditions which may induce sufficient leukocyte apoptosis without excessive necrosis, and under conditions which may maintain the biological integrity of the blood. The treated blood is subsequently re-infused into the subject.

The present invention comprises methods that reduce the edema in the ischemic penumbra of acute ischemic brain stroke patients, and/or reduce the edema in the area surrounding the infarct which may include ischemic tissue and the ischemic penumbra of acute ischemic brain stroke patients, as evaluated by a variety of diagnostic tools including MRI and Doppler imaging techniques.

The present invention is directed to reducing inflammation to cause a reduction in edema in the ischemic penumbra of acute ischemic brain stroke patients, and to reduce inflammation causing a reduction in edema in the area surrounding the infarct, which may include ischemic tissue and the ischemic penumbra of acute ischemic brain stroke patients.

The effects of blood, blood fractionate or other fluid which has absorbed a quantifiable absorbed-dose of ozone, when re-infused into a mammalian patient's body may include effects that reduce inflammation.

Reduction of inflammation may occur though a reduction in pro-inflammatory cytokines (e.g. interferon-gamma, TNF-gamma, IL-6 and IL-12) and/or an increase in anti-inflammatory cytokines (e.g. interleukin-4 and IL-10) released by immunomodulatory T cells. The effect of reducing inflammation may result in any number of clinical benefits including improving endothelial function including endothelial cellular repair or replacement. These results may lead to a reduction in edema in the ischemic penumbra.

Reduction of inflammation may occur though a reduction in pro-inflammatory cytokines (e.g. interferon-gamma, TNF-gamma, IL-6 and IL-12) and/or an increase in anti-inflammatory cytokines (e.g. interleukin-4 and IL-10) released by immunomodulatory T cells. The effect of reducing inflammation may result in any number of clinical benefits including improving endothelial function including endothelial cellular repair or replacement. These results may lead to a reduction in edema in the area surrounding the infarct which may include ischemic tissue and the ischemic penumbra.

The methods of the present invention provide for treatment of acute ischemic brain stroke, including a method of delivery of a measured amount of ozone and subsequent absorption of a quantifiable absorbed-dose of ozone by blood or derivatives thereof extracorporeally which, when reinfused autologously into a patient, may cause a reduction in CRP.

The methods of the present invention provide for treatment of acute ischemic brain stroke, including a method of delivery of a measured amount of ozone and subsequent absorption of a quantifiable absorbed-dose of ozone by blood or derivatives thereof extracorporeally which, when reinfused autologously into a patient, may cause a reduction in CRP sufficient to elicit clinical benefit. Clinical benefits may include reduction of inflammation, increasing blood flow through vasodilation and increasing blood flow through neovascularization.

The methods of the present invention are directed to increasing blood flow to the area surrounding the infarct which may include ischemic tissue and the ischemic penumbra of acute ischemic brain stroke patients, the method comprising connecting a subject to a device for withdrawing blood, withdrawing blood and delivering a measured amount of ozone to the blood under conditions which may maintain the biological integrity of the blood. The treated blood is subsequently re-infused into the subject.

The methods of the present invention are directed to increasing blood flow to the area surrounding the infarct which may include ischemic tissue and the ischemic penumbra of acute ischemic brain stroke patients as evaluated by a variety of diagnostic tools including MRI and Doppler imaging techniques.

The methods of the present invention are further directed to reducing inflammation causing an increase in blood flow to the area surrounding the infarct which may include ischemic tissue and the ischemic penumbra of acute ischemic brain stroke patients.

The methods of the present invention further provide for a treatment of blood, blood fractionate or other fluid, and the administration of this treated fluid in the treatment of acute ischemic brain stroke is to relax the vascular endothelium.

The present method is based upon extracorporeally subjecting an aliquot of a mammalian patient's blood, or the separated cellular fractions of the blood, or mixtures of the separated cells, including platelets, to a measured amount of ozone such that it absorbs a quantifiable absorbed-dose of ozone. On re-introduction of this autologous aliquot to the patient's body, the blood, blood fractionate or other fluid with a quantifiable absorbed-dose of ozone may have certain beneficial effects. One of these effects is to relax the endothelium. This relaxation may result from an increase in vasodilation (i.e. promotion of vasodilators or inhibition of vasoconstrictors) improving endothelial function including endothelial cellular repair or replacement, and improving blood flow yielding enhanced oxygenation. Thus, the methods of the present invention are further directed to promoting relaxation in the vascular endothelium, thereby causing a reduction in edema in the ischemic penumbra of acute ischemic brain stroke patients. Relaxation of the vascular endothelium may result from a reduction in edema in the area surrounding the infarct, which may include ischemic tissue and the ischemic penumbra of acute ischemic brain stroke patients.

The effects of blood, blood fractionate or other fluid which has absorbed a quantifiable absorbed-dose of ozone, when re-infused into a mammalian patient's body may include a relaxation of the vascular endothelium. These effects may result in an increase in vasodilation (i.e. promotion of vasodilators or inhibition of vasoconstrictors), improving endothelial function including endothelial cellular repair or replacement. These results may lead to a reduction in the edema in the ischemic penumbra of patients suffering from acute ischemic brain stroke, as well as in the area surrounding the infarct, which may include ischemic tissue and the ischemic penumbra of patients suffering from acute ischemic brain stroke.

The methods of the present invention are further directed to relaxing the vascular endothelium causing a reduction in edema in the ischemic penumbra of acute ischemic brain stroke patients, and/or relaxing the vascular endothelium causing a reduction in edema in the area surrounding the infarct which may include ischemic tissue and the ischemic penumbra of acute ischemic brain stroke patients, as evaluated by a variety of diagnostic tools including MRI and Doppler imaging techniques.

The methods of the present invention are further directed to relaxing the vascular endothelium to cause an increase in blood flow to the area surrounding the infarct which may include ischemic tissue and the ischemic penumbra of acute ischemic brain stroke patients.

The effects of blood, blood fractionate or other fluid which has absorbed a quantifiable absorbed-dose of ozone, when re-infused into a mammalian patient's body may include a relaxation of the vascular endothelium. These effects may result in an increase in vasodilation (i.e. promotion of vasodilators or inhibition of vasoconstrictors), improving endothelial function including endothelial cellular repair or replacement. These results may lead to an increase in blood flow to the area surrounding the infarct which may include ischemic tissue and the ischemic penumbra in patients suffering from acute ischemic brain stroke.

The methods of the present invention are, therefore, directed to relaxing the vascular endothelium causing an increase in blood flow to the area surrounding the infarct which may include ischemic tissue and the ischemic penumbra of acute ischemic brain stroke patients as evaluated by a variety of diagnostic tools including MRI and Doppler imaging techniques.

The methods of the present invention are directed to the re-introduction of an autologous aliquot of a mammalian patient's blood, blood fractionate or other fluid which has absorbed a quantifiable absorbed-dose of ozone may be through a variety of routes including intravenous, intramuscular and subcutaneous, or any combination thereof.

The methods of the present invention provide a treatment of acute ischemic brain stroke in a mammalian patient by treating blood by a discontinuous flow method. The method comprising connecting a subject to a device for withdrawing blood, withdrawing blood and delivering a measured amount of ozone to the blood under conditions which may maintain the biological integrity of the blood wherein the treated blood is subsequently re-infused into the patient.

The methods of the present invention further provide a treatment of acute ischemic brain stroke in a mammalian patient by treating blood, or a fraction thereof, including plasma or serum, by a discontinuous flow method. The method comprising connecting a subject to a device for withdrawing blood, withdrawing blood containing blood cells from the subject, separating a non-cellular fraction from the blood and delivering a measured amount of ozone to the fraction, under conditions which may maintain the biological integrity of the blood fraction. The treated fraction is subsequently recombined with the blood cells and re-infused into the subject.

The methods of the present invention provide a treatment that causes improvement in paralysis in patients suffering from acute ischemic brain stroke, that causes improvement in paralysis in patients suffering from acute ischemic brain stroke as evaluated by stroke scale assessment tools, that causes improvement in paralysis in patients suffering from acute ischemic brain stroke as evaluated by stroke scale assessment tools, and wherein clinical effectiveness is measured through statistical comparison with untreated stroke patients.

The methods of the present invention further provide a treatment that causes improvement in motor weakness in patients suffering from acute ischemic brain stroke, as may be evaluated by stroke scale assessment tools and/or wherein clinical effectiveness is measured through statistical comparison with untreated stroke patients.

The methods of the present invention further provide a treatment that causes improvement in loss of sensation in patients suffering from acute ischemic brain stroke, as may be evaluated by stroke scale assessment tools, and wherein clinical effectiveness may be measured through statistical comparison with untreated stroke patients.

The methods of the present invention provide a treatment that causes improvement in ocular and auditory functions in patients suffering from acute ischemic brain stroke, as may be evaluated by stroke scale assessment tools, and wherein clinical effectiveness may be measured through statistical comparison with untreated stroke patients.

The methods of the present invention provide a treatment that causes reduction in the severity of recurrent stroke in patients suffering from acute ischemic brain stroke, as may be evaluated by stroke scale assessment tools, and wherein clinical effectiveness may be measured through statistical comparison with untreated stroke patients.

The methods of the present invention further provide a treatment that causes improvement in cognitive function in patients suffering from acute ischemic brain stroke, as may be evaluated by stroke scale assessment tools, and wherein clinical effectiveness may be measured through statistical comparison with untreated stroke patients.

The methods of the present invention provide a treatment that causes improvement in verbal communication in patients suffering from acute ischemic brain stroke, as may be evaluated by stroke scale assessment tools, and wherein clinical effectiveness may be measured through statistical comparison with untreated stroke patients.

The methods of the present invention may further provide a treatment that causes re-attainment of independence in patients suffering from acute ischemic brain stroke as evaluated by stroke scale assessment tools and wherein clinical effectiveness is measured through statistical comparison with untreated stroke patients.

The methods of the present invention may further provide a treatment that causes improvement in the rate of stroke-free and/or overall survival in patients suffering from acute ischemic brain stroke, as evaluated through statistical comparison with untreated stroke patients, as may be evaluated through statistical comparison with untreated stroke patients.

The methods of the present invention may further provide a treatment of acute ischemic brain stroke wherein there is a shift from a pro-inflammatory state to an anti-inflammatory state of the vascular endothelium.

The methods of the present invention may further provide for relaxation of the vascular endothelium through the release of anti-inflammatory cytokines including interleukin-4 and interleukin-10 and TGF-gamma. The methods of the present invention may further provide for the relaxation of the vascular endothelium through the inhibition of pro-inflammatory cytokines including interferon-gamma, TNF-gamma, IL-1, IL-6 and IL-12.

The methods of the present invention may further provide a treatment for acute ischemic brain stroke by inhibiting vasoconstriction of the vascular endothelium. The methods of the present invention may further provide a treatment of acute ischemic brain stroke by promoting vasodilation of the vascular endothelium.

The methods of the present invention may further provide a treatment for acute ischemic brain stroke by causing the release of endothelium-derived relaxing factor, nitric oxide, prostacyclin or other related vasodilatory compounds.

The methods of the present invention may provide a treatment for acute ischemic brain stroke wherein there is an increase in oxygen delivered to the ischemic area. The methods of the present invention may further provide a treatment of acute ischemic brain stroke by promoting angiogenesis in the ischemic area.

### BRIEF DESCRIPTION OF DRAWINGS

To further clarify the present invention, specific embodiments thereof are illustrated in the appended drawings, which schematically illustrate what is currently considered the best mode for carrying out the invention;

FIG. 1 illustrates, in a schematic diagram, alternate methods of carrying out treatment of a fluid from a patient, comprising a continuous loop format and, alternatively, a discontinuous flow format.

### DETAILED DESCRIPTION OF THE EMBODIMENTS OF THE INVENTION

### Definitions

As used herein, “comprising,” “including,” “containing,” “characterized by,” and grammatical equivalents thereof are inclusive or open-ended terms that do not exclude additional, unrecited elements or method steps, but also include the more restrictive terms “consisting of” and “consisting essentially of.”

As used herein and in the appended claims, the singular forms, for example, “a”, “an”, and “the,” include the plural, unless the context clearly dictates otherwise. For example, reference to “a gas-fluid contacting device” includes a plurality of such devices, and reference, for example, to a “protein” is a reference to a plurality of similar proteins, and equivalents thereof.

An “ozone/oxygen admixture” refers to a concentration of ozone in an oxygen carrier gas. Various units of concentration utilized by those skilled in the art include: micrograms of ozone per milliliter of oxygen, parts (ozone) per million (oxygen) by weight (‘ppm’) and parts per million by volume (‘ppmv’). As a unit of concentration for ozone in oxygen, ppmv is defined as the molar ratio between ozone and oxygen. One ppmv ozone is equal to 0.00214 micrograms of ozone per milliliter of oxygen. Additionally, one ppm ozone equals 0.00143 micrograms of ozone per milliliter of oxygen. In terms of percentage ozone by weight, 1% ozone equals 14.3 micrograms of ozone per milliliter of oxygen. All units of concentration and their equivalents are calculated at standard temperature and pressure (i.e. 25° C. at 1 atmosphere).

“Delivered-ozone” is the amount of ozone contained within a volume of an ozone/oxygen admixture that is delivered to a fluid, and is synonymous with the delivery of a measured amount of ozone.

“Absorbed-ozone” is the amount of delivered-ozone that is actually absorbed and utilized by an amount of fluid, and is synonymous with a quantifiable absorbed dose of ozone.

“Residual-ozone” is the amount of delivered-ozone that is not absorbed such that:

Residual-ozone=delivered-ozone−absorbed-dose of ozone.

An “interface” is defined as the contact between a fluid and an ozone/oxygen admixture.

“Interface-time” is the time that a fluid resides within a gas-fluid contacting device and is interfaced with an ozone/oxygen admixture.

“Interface surface area” is defined as the dimensions of the surface within a gas-fluid contacting device over which a fluid flows and contacts an ozone/oxygen admixture.

“Elapsed-time” is the time that a fluid circulates throughout an ozone delivery system, including passage through one or more gas-fluid contacting devices, connecting tubing and an optional reservoir.

“Ozone-inert” materials are defined as construction materials of an ozone delivery system that do not react with ozone in a manner that introduces contaminants or deleterious byproducts of oxidation of the construction materials into a fluid, and which do not absorb ozone.

“Non-reactive” is defined as not readily interacting with other elements or compounds to form new chemical compounds.

“Measured-data” is defined as information collected from various measuring components (such as an inlet ozone concentration monitor, exit ozone concentration monitor, gas flow meter, fluid pump, data acquisition device, humidity sensor, temperature sensor, pressure sensor, absorbed oxygen sensor) throughout the system.

“Calculated-data” is defined as the mathematical treatment of measured-data by a data acquisition device.

“Absorption of ozone by a biological fluid” is defined as the phenomenon wherein ozone reacts with the fluid being treated by a variety of mechanisms, including oxidation. Regardless of the mechanism involved, the reaction occurs instantaneously, and the products of this reaction include oxidative products, of which lipid peroxides are an example.

A “biological fluid” is defined as a composition originating from a biological organism of any type. Examples of biological fluids include blood, blood products and other fluids, such as saliva, urine, feces, semen, milk, tissue, tissue samples, homogenized tissue samples, gelatin, and any other substance having its origin in a biological organism. Biological fluids may also include synthetic materials incorporating a substance having its origin in a biological organism, such as a vaccine preparation containing alum and a virus (the virus being the substance having its origin in a biological organism), cell culture media, cell cultures, viral cultures, and other cultures derived from a biological organism.

A “blood product” is defined as including blood fractionates and therapeutic protein compositions containing proteins derived from blood. Fluids containing biologically active proteins other than those derived from blood may also be treated by the method.

“In vivo” use of a material or compound is defined as the introduction of a material or compound into a living human, mammal or vertebrate.

“In vitro” use of a material or compound is defined as the use of the material or compound outside a living human, mammal or vertebrate, where neither the material nor compound is intended for reintroduction into a living human, mammal or vertebrate. An example of an in vitro use would be the analysis of a component of a blood sample using laboratory equipment.

“Ex vivo” use of a process is defined as using a process for treatment of a biological material such as a blood product outside of a living human, mammal or vertebrate. For example, removing blood from a human and subjecting that blood to a method to treat acute ischemic brain stroke is defined as an ex vivo use of that method if the blood is intended for reintroduction into that human or another human. Reintroduction of the human blood into that human or another human would be an in vivo use of the blood, as opposed to an ex vivo use of the method.

“Extracorporeal” is defined as a state wherein blood or blood fractionate is treated outside (ex vivo) of the body, for example, in the delivery of a measured amount of ozone to a sample of patient's blood.

“Synthetic media” is defined as an aqueous synthetic blood or blood product storage media.

A “pharmaceutically-acceptable carrier” or “pharmaceutically-acceptable vehicle” is defined as any liquid including water, saline, a gel, salve, solvent, diluent, fluid ointment base, liposome, micelle and giant micelle, which is suitable for use in contact with a living animal or human tissue without causing adverse physiological responses, and which does not interact with the other components of the composition in a deleterious manner.

“Biologically active” is defined as capable of effecting a change in the living organism or component thereof.

The “biological integrity of a biological fluid” is defined as a quality or state of a fluid that, subsequent to the method of treating for acute ischemic brain stroke described herein, sufficiently maintains its functionality upon re-infusion into a mammalian patient.

“Acute ischemic brain stroke” is defined as a blockage in a blood vessel that stops the flow of blood and deprives the surrounding brain tissue of oxygen.

“Edema” is defined as a condition of abnormally large fluid volume in the circulatory system or in tissues between the body's cells (interstitial spaces).

“C-reactive protein” is defined as a liver-synthesized, acute phase reactant protein regarded as a marker of acute inflammation capable of activating the classical compliment pathway and opsonizing ligands for phagocytosis.

The present invention describes methods for therapeutic treatment of acute ischemic brain stroke which are based on the delivery of a measured amount of ozone to a sample of a patient's blood, blood fractionate or other fluid extracorporeally through the use of an ozone delivery system. A quantifiable absorbed-dose of ozone absorbed by the fluid is subsequently re-infused into the same patient. This autologous blood sample, which contains a quantifiable absorbed-dose of ozone, can effect improvement in any condition caused by stroke including reduction in edema associated with the ischemic penumbra, improvement in impaired blood flow to the area surrounding the infarct which may include ischemic tissue and the ischemic penumbra, relaxation of the vascular endothelium, and reduction of inflammation. Positive treatment outcomes may be measured and may include improvement in paralysis, visual and auditory skills, cognitive function, re-attainment of independence, stroke-free survival, relapse frequency and severity, and overall post-stroke survival.

The methods of the present invention provide a novel approach in the treatment of acute ischemic brain stroke, including a method of delivery of a measured amount of ozone and subsequent absorption of a quantifiable absorbed-dose of ozone by blood or derivatives thereof extracorporeally, which may cause sufficient leukocyte apoptosis without excessive necrosis necessary to elicit clinical benefit following reinfusion of the autologous fluid to the patient.

The methods of the present invention also provide novel treatments of acute ischemic brain stroke, including a method of delivering a measured amount of ozone, and subsequent absorption of a quantifiable absorbed-dose of ozone, by blood or derivatives thereof extracorporeally which, when reinfused autologously into a patient, may cause a reduction in CRP sufficient to elicit clinical benefit.

The methods of the present invention provide novel treatment of acute ischemic brain stroke, including a method of delivering a measured amount of ozone, and subsequent absorption of a quantifiable absorbed-dose of ozone by blood or derivatives thereof, extracorporeally, which, upon reinfusion, may affect relaxation of vascular endothelium and may involve release of vasodilators including nitric oxide and prostacyclins, sufficient to elicit clinical benefit.

The methods of the present invention provide for a treatment of blood, blood fractionate or other fluid, and the use of this treated blood, blood fractionate or other fluid in the treatment of acute ischemic brain stroke in a mammalian patient by administration to the patient of such treated blood, blood fractionate or other fluid.

The methods of the present invention provide are based upon extracorporeally subjecting an aliquot of a mammalian patient's blood, or the separated cellular fractions of the blood, or mixtures of the separated cells, including platelets, to a measured amount of ozone such that it absorbs a quantifiable absorbed-dose of ozone. On re-introduction of this autologous aliquot to the patient's body, the blood, blood fractionate or other fluid with a quantifiable absorbed-dose of ozone may have certain beneficial effects. These effects may result in the improvement in any condition caused by stroke, including the reduction in edema in ischemic penumbra, improvement in impaired blood flow to the area surrounding the infarct which may include ischemic tissue and the ischemic penumbra, relaxation of the vascular endothelium and reduction of inflammation.

The effect of blood, or blood derivatives thereof, which has absorbed a quantifiable absorbed-dose of ozone, may be the induction of sufficient leukocyte apoptosis without excessive necrosis necessary to elicit an anti-inflammatory response when reinfused autologously into a patient. The induction of apoptosis without excessive necrosis in the leukocyte fraction of the blood treated may be evaluated by a number of diagnostic methods including light microscopy with nuclear stains, electrophoretic analysis of DNA fragmentation, TUNEL analysis and multiparameter flow cytometry.

An effect of blood or blood derivative thereof which has absorbed a quantifiable absorbed-dose of ozone, may result in the reduction of CRP when reinfused autologously into a patient and elicit clinical benefit including an anti-inflammatory response, neovascularization and vasodilation.

The effects of blood, blood fractionate or other fluid which has absorbed a quantifiable absorbed-dose of ozone, when reinfused into a mammalian patient's body may include effects that reduce the edema in the ischemic penumbra in acute ischemic brain stroke patients. This reduction can be evaluated by a variety of diagnostic tools including MRI and Doppler imaging techniques.

In addition, the effects of blood, blood fractionate or other fluid which has absorbed a quantifiable absorbed-dose of ozone, when re-infused into a mammalian patient's body may include effects that increase blood flow to the area surrounding the infarct which may include ischemic tissue and the ischemic penumbra in patients suffering from acute ischemic stroke. This reduction can be evaluated by a variety of diagnostic tools including MRI and Doppler imaging techniques.

Moreover, the effects of blood, blood fractionate or other fluid which has absorbed a quantifiable absorbed-dose of ozone, when re-infused into a mammalian patient's body may include effects that relax the vascular endothelium in patients suffering from acute ischemic stroke. This relaxation may result from an increase in vasodilation (i.e. promotion of vasodilators or inhibition of vasoconstrictors) improving endothelial function including endothelial cellular repair or replacement, and improving blood flow yielding enhanced oxygenation.

The effects of blood, blood fractionate or other fluid which has absorbed a quantifiable absorbed-dose of ozone, when re-infused into a mammalian patient's body may include effects that include reduction of inflammation. Reduction of inflammation may occur though a reduction in pro-inflammatory cytokines (e.g. interferon-gamma, TNF-gamma, IL-6 and IL-12) and/or an increase in anti-inflammatory cytokines (e.g. interleukin-4 and IL-10) released by immunomodulatory T cells. The effect of reducing inflammation may result in any number of clinical benefits including improvement in blood flow yielding enhanced oxygenation.

The effect of treated blood or blood derivative thereof with ozone by the present method to induce apoptotic leukocytes without excessive necrosis, when re-infused into a mammalian patient's body may include effects that include reduce of inflammation. Reduction of inflammation may occur though a reduction in pro-inflammatory cytokines (e.g. interferon-gamma, TNF-gamma, IL-6 and IL-12) and/or an increase in anti-inflammatory cytokines (e.g. interleukin-4 and IL-10) released by immunomodulatory cells. The effect reducing inflammation may result in any number of clinical benefits in the treatment of acute ischemic stroke including improvement in blood flow yielding enhanced oxygenation.

The methods of the present invention are based upon extracorporeally subjecting an aliquot of a mammalian patient's blood, or the separated cellular fractions of the blood, or mixtures of the separated cells, including platelets, to a measured amount of ozone such that it absorbs a quantifiable absorbed-dose of ozone. On re-introduction of this autologous aliquot to the patient's body, the blood, blood fractionate or other fluid with a quantifiable absorbed-dose of ozone which may affect improvement in any condition caused by stroke including: paralysis, motor weakness, visual and auditory skills, cognitive function, re-attainment of independence, stroke-free survival, relapse frequency and severity, and overall survival.

The effects of blood, blood fractionate or other fluid which has absorbed a quantifiable absorbed-dose of ozone, when re-infused into a mammalian patient's body which may affect improvement in any condition caused by stroke including improvement in paralysis, motor weakness, visual and auditory skills, cognitive function, re-attainment of independence, stroke-free survival, relapse frequency and severity, and overall survival which may be evaluated by stroke scale assessment tools.

In addition, the effects of blood, blood fractionate or other fluid which has absorbed a quantifiable absorbed-dose of ozone, when re-infused into a mammalian patient's body which may affect improvement in any condition caused by stroke may be evaluated by stroke scale assessment tools and wherein clinical effectiveness is measured through statistical comparison with untreated stroke patients.

The methods of the present invention are based upon extracorporeally subjecting an aliquot of a mammalian patient's blood, or the separated cellular fractions of the blood, or mixtures of the separated cells, including platelets, to a measured amount of ozone such that it absorbs a quantifiable absorbed-dose of ozone. On re-introduction of this autologous aliquot to the patient's body, the blood, blood fractionate or other fluid with a quantifiable absorbed-dose of ozone may result in the improvement in any condition caused by stroke. Re-introduction of this autologous aliquot to a mammalian patient may be through a variety of routes including intravenous, intramuscular and subcutaneous, or any combination thereof.

An ozone delivery system utilized in the treatment of acute ischemic brain stroke delivers a measured amount of an ozone/oxygen admixture and is able to measure, control, report and differentiate between the delivered-ozone and absorbed-dose of ozone. The system provides a controllable, measurable, accurate and reproducible amount of ozone that is delivered to a controllable, measurable, accurate and reproducible amount of a biological fluid and controls the rate of ozone absorption by the fluid resulting in a quantifiable absorbed-dose of ozone used in the treatment of acute ischemic brain stroke.

The ozone delivery system may accomplish this by using a manufacturing component, control components, measuring components, a reporting component and calculating component (such as an ozone generator, gas flow meter, fluid pump, variable pitch platform, data acquisition device, inlet ozone concentration monitor, and exit ozone concentration monitor) that cooperate to manufacture and deliver a measured, controlled, accurate and reproducible amount of ozone (i.e., the delivered-ozone) to a fluid through the use of one or more gas-fluid contacting devices that provide for the interface between the ozone/oxygen admixture and fluid.

Using control components, measuring components, a reporting component and calculating components (such as a gas flow meter, fluid pump, variable pitch platform, data acquisition device, inlet ozone concentration monitor, and exit ozone concentration monitor) that cooperate, the system may instantly differentiate the delivered-ozone from the absorbed-dose of ozone.

A particularly suitable ozone delivery system that may be used in carrying out the methods of the present invention is disclosed in U.S. Pat. No. 7,736,494 and co-pending application Ser. No. 12/813,371, the contents of which are incorporated herein in their entirety. The disclosed ozone delivery system is particularly and uniquely constructed such that all ozone-contacting surfaces of the device are made of ozone-inert material so that the amount of ozone that is actually absorbed by the biological fluid being treated is accurately determinable. That is, by virtue of being constructed with ozone-inert materials in all ozone-contacting surfaces, no ozone is absorbed by the device itself, and the determination of the amount of ozone absorbed by the biological fluid is not inaccurately reflected as a result of ozone being absorbed by any structure of the device. The ozone delivery system utilizes measuring components, reporting components and calculating components (such as an inlet ozone concentration monitor, exit ozone concentration monitor, gas flow meter, fluid pump, data acquisition device) that cooperate together to determine certain calculated-data including the delivered-ozone, the residual-ozone and the absorbed-dose of ozone.

Delivered-ozone is an amount of ozone calculated by multiplying the measured volume of ozone/oxygen admixtures, as reported by gas flow meters, by the measured concentration of ozone within the ozone/oxygen admixture as it enters the gas-fluid contacting device, as reported by the inlet ozone concentration monitor. The measured volume of ozone/oxygen admixtures is calculated by multiplying the measured gas flow reported by gas flow meters, by the elapsed-time.

Residual-ozone is an amount of ozone calculated by multiplying the measured volume of ozone/oxygen admixtures, as reported by gas flow meters, by the measured concentration of ozone within the ozone/oxygen admixture exiting the gas-fluid contacting device, as reported by the exit ozone concentration monitor. The measured volume of ozone/oxygen admixtures is calculated by multiplying the measured gas flow reported by gas flow meters, by the elapsed-time.

The quantifiable absorbed-dose of ozone is an amount of ozone calculated by subtracting the amount of residual-ozone from the amount of delivered-ozone. The quantifiable absorbed-dose of ozone may range from 1 to 10,000,000 micrograms per milliliter of fluid, and may be between 1 and 10,000 μg per milliliter of fluid.

All measured-data, including measured data from the gas flow meters, inlet and exit ozone concentration monitors, the fluid pump, temperature sensors, pressure sensors, absorbed oxygen sensor and humidity sensors are reported to a data acquisition device. The data acquisition device has instant, real-time reporting, calculating and data storing capabilities to process all measured data. The data acquisition device may use any measured data or any combination of measured data as variables to produce calculated-data. Examples of calculated-data may include delivered-ozone, residual-ozone, absorbed-dose of ozone, absorbed-dose of ozone per unit volume of fluid, and the quantifiable absorbed-dose of ozone per unit volume of fluid per unit time.

An ozone delivery system particularly suitable to the present invention includes an ozone generator for the manufacture and control of a measured amount of an ozone/oxygen admixture and where the admixture volume contains the delivered-ozone. A commercially available ozone generator capable of producing ozone in a concentration range between 10 and 3,000,000 ppmv of ozone in an ozone/oxygen admixture may be employed. Ozone/oxygen admixture concentrations entering the gas-fluid contacting device are instantly and constantly measured in real time, through an inlet ozone concentration monitor that may utilize UV absorption as a detection methodology. A flow meter controls and measures the delivery of the delivered-ozone in an ozone/oxygen admixture to the gas-fluid contacting device at a specified admixture flow rate. Ozone/oxygen admixture flow rates are typically in the range between 0.1 and 5.0 liters per minute.

Measurement of the humidity of the ozone/oxygen admixture delivered to the gas-fluid contacting device may be included through the use of a humidity sensor. A humidity sensor port may be provided in the ozone/oxygen admixture connecting tubing, however, it can be placed in a variety of locations. For example, the humidity sensor may be located in the connecting tubing prior to the admixture's entrance into gas-fluid contacting device.

Measurement of the temperature within the gas-fluid contacting device during the interface-time may be provided by inclusion of a temperature sensor port in the gas fluid contacting device through which a temperature sensor may be inserted. The temperature at which ozone/oxygen admixtures interface fluids ranges from 4° C. to 100° C., and may be performed at ambient temperature, 25° C., for example. The temperature at which the interface occurs can be controlled by placing the gas-fluid contacting device, optional reservoir, and both gas and fluid connecting tubing in a temperature controlled environment, and/or by the addition of heating or cooling elements to the gas-fluid contact device.

Measurement of the pressure within the gas-fluid contacting device during the interface-time is provided by inclusion of a pressure sensor port in the gas-fluid contacting device through which a pressure sensor may be inserted. The pressure at which an ozone/oxygen admixtures interfaces with a fluid ranges from ambient pressure to 50 psi and may be performed between ambient pressure and 3 psi, for example. A pressure sensor port may be provided in each gas-fluid contacting device to measure and report the pressure at which the interface occurs.

The concentration of the ozone/oxygen admixtures exiting the gas-fluid contacting device and where the admixture volume contains the residual-ozone, are instantly and constantly measured in real time through an exit ozone concentration monitor that may utilize UV absorption as a detection methodology.

A fluid pump controls and measures the flow rate of the fluid delivered to the gas-fluid-contacting device at a specified fluid flow rate. Fluid flow rates through the gas-fluid contacting device typically will range from 1 ml to 100 liters per minute, and for example, may be between 1 ml to 10 liters per minute. The fluid is generally contained within a closed-loop design and may be circulated through the gas-fluid contacting device once or multiple times.

Measurement of the amount of oxygen absorbed into a fluid while it interfaces with the ozone/oxygen admixture within the gas-fluid contacting device may be provided through the use of an absorbed oxygen sensor. The sensor is inserted within the absorbed oxygen sensor port located in the tubing as it exits the gas-fluid contacting device. Measurement of absorbed oxygen may be recorded in various units, including ppm, milligrams/liter or percent saturation.

The system may also include a fluid access port for fluid removal. The port may generally be located in a tubing member after the fluid exits through the fluid exit port of the gas-fluid contacting device and prior to an optional reservoir.

A data acquisition device, such as a DAQSTATION (Yokogawa), for example, reports, stores and monitors data instantly and in real-time, and performs various calculations and statistical operations on data acquired. Data is transmitted to the data acquisition device through data cables, including data from ozone concentration monitors, flow meters, a humidity sensor, temperature sensors, pressure sensors, a fluid pump and an absorbed oxygen sensor.

Calculated data in carrying out the methods of the present invention include delivered-ozone, residual-ozone and the absorbed-dose of ozone. Measurement of the volume of the ozone/oxygen admixture delivered can be calculated though data provided from the flow meter and the time measurement capability of the data acquisition device. Measurement of the volume of fluid delivered to the gas-fluid contacting device can be calculated by the data acquisition device utilizing fluid flow rate data transmitted from the fluid pump.

The elapsed-time can be measured and controlled through the data acquisition device. The elapsed-time that the fluid circulates through the apparatus including the gas-fluid contacting device and is interfaced with an ozone/oxygen admixture can vary, generally for a duration of up to 120 hours. The interface-time may also be measured by the time measuring capacity of the data acquisition device. The interface-time between a fluid and an ozone/oxygen admixture may be controlled through a composite of controls. These controls include the angle of the gas fluid contacting device, the fluid flow rate via fluid pump, and the time controlling capacity of the data acquisition device. The interface-time may vary in duration of up to 720 minutes, and generally within duration of up to 120 minutes.

Controllable variables for an ozone delivery system may include delivered amounts and concentrations of ozone in the entering ozone/oxygen admixtures, fluid flow rates, admixture flow rates, temperature in the gas-fluid contacting device, interface-time between fluid and admixture; and, the elapsed-time that the fluid may circulate through the apparatus and interface with an ozone/oxygen admixture.

Measurable variables may include: ozone/oxygen admixture flow rates, amounts and concentrations of ozone in the entrance and exit ozone/oxygen admixtures, fluid flow rates, temperature and pressure in the gas-contacting device, humidity of the entrance admixture to the gas-fluid contacting device, absorbed oxygen by the fluid, interface-time and elapsed-time.

Data representing controllable variables and measurable variables acquired by the apparatus allows for a variety of calculations including: delivered-ozone, residual-ozone, absorbed-dose of ozone, absorbed-dose of ozone per unit volume of fluid, and the absorbed-dose of ozone per unit volume of fluid per unit time.

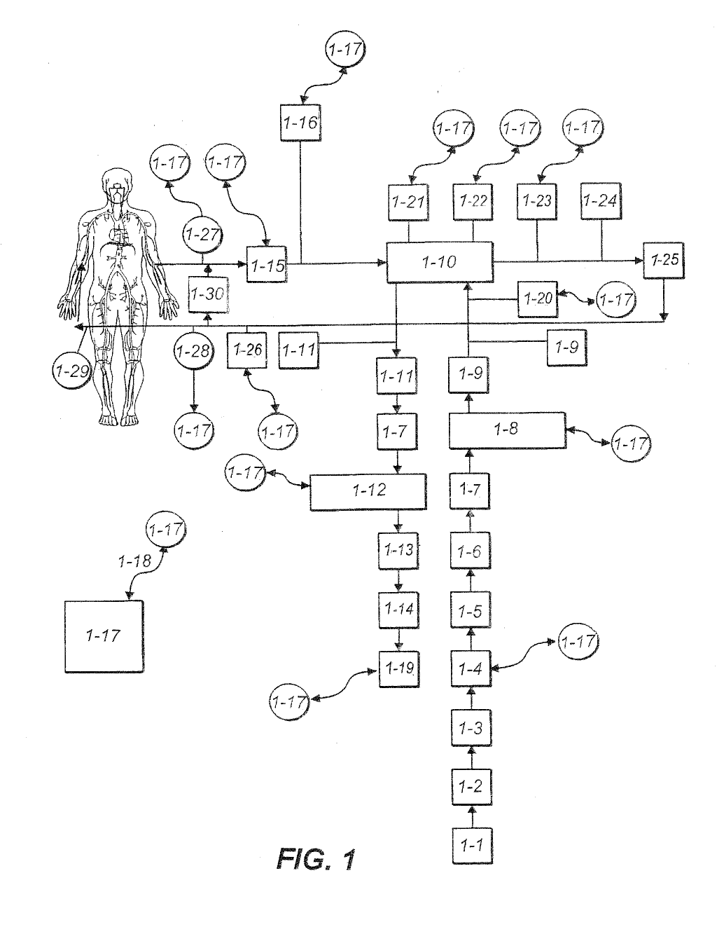

FIG. 1 schematically illustrates an embodiment of the present invention where fluid that has been taken from a subject is extracorporeally interfaced with an ozone/oxygen admixture. In general, blood may be circulated in a discontinuous manner where a fluid (e.g., an aliquot of blood) has been removed from a patient and is introduced into an ozone delivery system through a common reservoir, and is recirculated in a closed loop format. Alternatively, fluid may be circulated in a continuous loop format in a venovenous extracorporeal exchange format. As an example, this continuous loop can be established through venous access of the antecubital veins of both right and left arms. Prior to establishing a discontinuous closed loop format, blood from the patient may be anticoagulated with citrate or any other suitable anticoagulant before being introduced in to the reservoir. For an extracorporeal continuous loop circuit, a patient may optionally be anticoagulated with heparin or any other suitable anticoagulant known to those skilled in the art.

For the gas flow in either the discontinuous format or continuous loop system, oxygen flows from a pressurized cylinder (1-1), through a regulator (1-2), through a particle filter (1-3) to remove particulates, through a flow meter (1-4) where the oxygen and subsequent ozone/oxygen admixture flow rate is controlled and measured. The oxygen proceeds through a pressure release valve (1-5), through an ozone generator (1-6) where the concentration of the ozone/oxygen admixture is manufactured and controlled and where the admixture volume includes the delivered-ozone. The ozone/oxygen admixture flows through an optional moisture trap (1-7), to reduce moisture.

The admixture proceeds through an inlet ozone concentration monitor (1-8) that measures and reports the inlet ozone concentration of the ozone/oxygen admixture that contains the delivered-ozone. This real-time measurement may be based on ozone's UV absorption characteristics as a detection methodology. The ozone/oxygen admixture then passes through a set of valves (1-9) used to isolate a gas-fluid contacting device for purging of gases. The ozone/oxygen admixture may pass an optional humidity sensor (1-20) where humidity may be measured and recorded, and into a gas-fluid contacting device (1-10) where it interfaces with fluid. The interface-time between fluid and ozone/oxygen admixture may be controlled through adjustment of a variable pitch platform, a fluid pump and the time controlling capacity of the data acquisition device.

The interface-time may then be measured by the data acquisition device (1-17). Temperature (1-21) and pressure (1-22) may be measured by the use of optional temperature and pressure sensors, respectively, inserted into their respective ports. The resultant ozone/oxygen admixture containing the residual-ozone exits the gas-fluid contacting device and flows through the exit purge valves (1-11), through a moisture trap (1-7), through an exit ozone concentration monitor (1-12), which may utilize a similar detection methodology as the inlet ozone concentration monitor (1-8), that measures and reports the exit ozone/oxygen admixture concentration. The exiting ozone/oxygen admixture then proceeds through a gas drier (1-13), through an ozone destructor (1-14) and a flow meter (1-19).

In the fluid flow for the discontinuous format, blood is introduced into the reservoir (1-30). In the continuous loop system, intravenous blood flows from the patient through tubing through a pressure gauge (1-27) which monitors the pressure of the blood flow exiting the patient. Generally, the pressure of the blood exiting the patient ranges from a negative pressure of 100-200 mm Hg, and may be between a negative pressure of 150 and 200 mm Hg, with a maximum cutoff pressure of minus 250 mm Hg. In either format, the blood flows through a fluid pump (1-15) and is optionally admixed with heparin or other suitable anticoagulant as provided by an optional heparin pump (1-16).

The blood then passes through the gas-fluid contacting device (1-10) where it interfaces with the ozone/oxygen admixture containing the delivered-ozone. Ports for the insertion of sensors may be located in the gas-fluid contacting device for the measurement of temperature and pressure, respectively. After interfacing with the ozone/oxygen admixture, the fluid exits into tubing that may contain a port for an optional absorbed oxygen sensor (1-23) followed by a fluid access port (1-24). The blood continues through an air/emboli trap (1-25) that removes any gaseous bubbles or emboli, and the blood then continues through a fluid pump (1-26).

In a discontinuous format, the blood is then directed back into the reservoir (1-30) any may continue in a recirculating mode, passaging as often as required. In the continuous loop format, the blood is directed into a pressure gauge (1-28) which monitors the pressure of the blood flow before returning the fluid to the patient. Generally, the pressure of the blood entering the patient ranges from a pressure of 100-200 mm Hg, and may be between 150 and 200 mm Hg, with a maximum cutoff pressure of 250 mm Hg. The blood continues through a priming fluid access port (1-29) that allows for the removal of the priming fluid from the extracorporeal loop. The blood is then re-infused directly into the patient.

A data acquisition device (1-17), such as a DAQSTATION (Yokogawa), for example, has time measurement capabilities, reports, stores and monitors data instantly and in real-time, and performs various calculations and statistical operations on data acquired. All data is transmitted to the data acquisition device through data cables (1-18), including: data from ozone concentration monitors (1-8) and (1-12), flow meters (1-4) and (1-19), humidity sensor (1-20), temperature sensor (1-21), pressure sensor (1-22), fluid pumps (1-15) and (1-26), pressure gauges (1-27) and (1-28), and absorbed oxygen sensor (1-23). The elapsed time, a composite of both the interface time and the period of time that the fluid circulates through the other elements of the apparatus can be measured and controlled through the data acquisition device (1-17).

Other possible configurations for an extracorporeal blood circuit known to those skilled in the art are included within the scope of this disclosure.

One or more gas-fluid contacting devices may be included in an ozone delivery system to increase the surface area of a fluid to be treated allowing for an increase in the mass transfer efficiency of the ozone/oxygen admixture. Gas-fluid contacting devices may encompass the following properties: closed and isolated from the ambient atmosphere, gas inlet and outlet ports for the entry and exit of ozone/oxygen admixtures, fluid inlet and outlet ports for the entry and exit of a fluid, components (temperature sensor, pressure sensor and data acquisition device) for the measurement and reporting of temperature and pressure within a gas-fluid contacting device, generation of a thin film of the fluid as it flows within a gas-fluid contacting device and construction from ozone-inert construction materials including, quartz, ceramic composite, borosilicate, stainless steel, PFA and PTFE.

Gas-fluid contacting devices include designs that encompass surfaces that may be horizontal or approaching a horizontal orientation. These surfaces may include ridges, indentations, undulations, etched surfaces or any other design that results in a contour change and furthermore, may include any pattern, regular or irregular, that may disrupt the flow, disperse the flow or cause turbulence. These surfaces may or may not contain holes through which a fluid passes through. The surface of the structural elements may have the same or different pitches. Designs of gas-fluid contacting devices may include those that involve one or more of the same shaped surfaces or any combination of different surfaces, assembled in any combination of ways to be encompassed within the device which may include cones, rods, tubes, flat and semi-flat surfaces, discs and spheres.

The interface between an ozone/oxygen admixture and a fluid may be accomplished by the use of a gas-fluid contact device that generates a thin film of the fluid that interfaces with the ozone-oxygen admixture as it flows through the device. One of skill in the art will appreciate that generation of any interface that increases the surface area of the fluid and thereby maximizes the contact between a fluid and an admixture, may be used. Additional examples include the generation of an aerosol through atomization or nebulization.

The interface-time within a gas-fluid contacting device is measurable, controllable, calculable and reportable. Furthermore, the interface-time may be for duration of up to 720 minutes, generally however, for duration of up to 120 minutes. Following the interface-time, the fluid exits the gas-fluid contacting device containing the quantifiable absorbed-dose of ozone. The elapsed-time, a composite of both the interface-time and the time for circulation of a fluid through other elements of an ozone delivery system is also measurable, controllable, calculable and reportable. This elapsed-time is for duration of up to 120 hours.

The pressure at the interface between fluid and ozone/oxygen admixture within a gas-fluid contacting device may be measured. Measurement of pressure within the device may be accomplished through the use of a pressure sensor inserted at the pressure port of the gas-fluid contacting device. The pressure at which an ozone/oxygen admixture interfaces with a fluid ranges from ambient pressure to 50 psi and may be performed between ambient pressure and 3 psi.

The temperature within a gas-fluid contacting device may be controlled by housing the device such that the connecting tubing containing both gas and fluid and an optional reservoir are maintained in a controlled temperature environment. A flow hood that provides for temperature regulation is an example of a controlled temperature environment. Alternatively, the addition of heating or cooling elements to the gas-fluid contact device may provide for the control of temperature. Measurement of temperature within the device may be accomplished through the use of a temperature sensor inserted at the temperature port of a gas-fluid contacting device. The temperature at which ozone/oxygen admixtures interface fluids ranges from 4° to 100° C., and may be performed at ambient temperature, 25° C., for example.

Gas-fluid contacting devices may be utilized individually or in conjunction with other such devices, whether they are similar or dissimilar in construction, design or orientation. In the event that multiple devices are utilized, either of the same design, or a combination of different gas-fluid contacting devices of different designs, these devices may be arranged one after the other in succession (in series), making a single device out of multiple individual contact devices.

In a series configuration of devices, a fluid flowing through the different contact devices flows in series, from the fluid exit port of one contact device to the fluid entrance port of the next, until passing through all the devices. The ozone/oxygen admixture may flow in a number of arrangements. In one example, the ozone/oxygen admixture flows through different contact devices in series, from the admixture exit port of one contact device to the admixture entrance port of the next. As an alternative example, the ozone/oxygen admixture may flow directly from the admixture source to the entrance port of each different contact device. Another alternative is a combination of the foregoing examples where the ozone/oxygen admixture flows from the exit port of some devices to the entrance port of other devices and in addition, to the entrance of some devices directly from the admixture source. In the event that multiple devices are utilized, the resultant fluid from the terminal device can either be collected or returned to the original device and recirculated.

When arranged in series with other contact devices, interface time between the fluid and ozone/oxygen admixture is controllable, and can be adjusted based on the individual pitch chosen for each device in series, or by adding additional devices to the series. The overall interface surface area will range from 0.01 m² for an individual device, and upwards based on the number of devices serially utilized.

### Example 1

An example of data measured and calculated by the ozone delivery system that utilizes a fluid target described herein is included in Table 1. Newborn Calf Serum commercially obtained was utilized as the target fluid. The variable pitch device with variable pitch platform as illustrated and disclosed in U.S. Pat. No. 7,736,494 were used as the gas-fluid contacting device. The following initial conditions were utilized; 300 ppmv ozone inlet concentration, 145 ml initial fluid volume, 1000 ml per minute gaseous flow rate, 189 ml per minute fluid flow rate counter current to the ozone/oxygen admixture flow. Incremental reductions in fluid volume are due to sampling of fluid through the fluid access port.

TABLE 1
NEWBORN CALF SERUM
MEASURED VARIABLES

| Elapsed-time | | | | Average Inlet Ozone | Average Exit Ozone |
|---|---|---|---|---|---|
| (5 min | Fluid Volume | Gas Flow Rate | Fluid Flow Rate | Concentration | Concentration |
| intervals) | (milliliters) | (liters/minute) | (liters/minute) | (ppmv) | (ppmv) |
| 5 | 145 | 0.998 | 0.189 | 305.2 | 38.2 |
| 10 | 143 | 0.972 | 0.189 | 361.5 | 40.4 |
| 15 | 141 | 1.000 | 0.189 | 312.7 | 20.6 |
| 20 | 139 | 1.000 | 0.189 | 314.0 | 37.3 |

CALCULATED VARIABLES

| | Average | | | | |
|---|---|---|---|---|---|
| | Differential Ozone | Delivered- | Residual- | Ozone-Absorbed | Absorbed-dose |
| Elapsed-time | Concentration | ozone | ozone | per Interval | of Ozone |
| (minutes) | (ppmv) | (ug) | (ug) | (ug) | (ug) |
| 5 | 267.0 | 3.26E+03 | 4.08E+02 | 2.86E+03 | 2.86E+03 |
| 10 | 321.1 | 7.02E+03 | 8.28E+02 | 3.34E+03 | 6.20E+03 |
| 15 | 292.1 | 1.04E+04 | 1.06E+03 | 3.12E+03 | 9.32E+03 |
| 20 | 276.7 | 1.37E+04 | 1.46E+03 | 2.96E+03 | 1.23E+04 |

### Example 2

An additional example of data measured and calculated by the system described herein is in Table 2 below. Newborn Calf Serum commercially obtained was utilized as the target fluid. The variable pitch device with variable pitch platform as illustrated and disclosed in U.S. Pat. No. 7,736,494 were used as the gas-fluid contacting device. The following initial conditions were utilized; 600 ppmv ozone inlet concentration, 137 ml initial fluid volume, 1000 ml per minute gaseous flow rate, 189 ml per minute fluid flow rate counter current to the ozone/oxygen admixture flow. Incremental reductions in fluid volume are due to sampling of fluid through the fluid access port.

TABLE 2
NEWBORN CALF SERUM
MEASURED VARIABLES

| Elapsed-time | | | | Average Inlet Ozone | Average Exit Ozone |
|---|---|---|---|---|---|
| (5 minute | Fluid Volume | Gas Flow Rate | Fluid Flow Rate | Concentration | Concentration |
| intervals) | (milliliters) | (liters/minute) | (liters/minute) | (ppmv) | (ppmv) |
| 5 | 137 | 1.000 | 0.189 | 604.2 | 72.0 |
| 5 | 135 | 1.000 | 0.189 | 609.6 | 63.5 |
| 5 | 133 | 1.000 | 0.189 | 606.6 | 70.8 |
| 5 | 131 | 1.000 | 0.189 | 605.3 | 71.7 |

CALCULATED VARIABLES

| | Average | | | | |
|---|---|---|---|---|---|
| | Differential Ozone | Delivered- | Residual- | Ozone Absorbed | Absorbed-dose |
| Elapsed-time | Concentration | ozone | ozone | per Interval | of ozone |
| (minutes) | (ppmv) | (ug) | (ug) | (ug) | (ug) |
| 5 | 532.2 | 6.47E+03 | 7.70E+02 | 5.69E+03 | 5.69E+03 |
| 10 | 546.1 | 1.30E+04 | 1.45E+03 | 5.84E+03 | 1.15E+04 |
| 15 | 535.8 | 1.95E+04 | 2.21E+03 | 5.73E+03 | 1.73E+04 |
| 20 | 533.6 | 2.60E+04 | 2.98E+03 | 5.71E+03 | 2.30E+04 |

### Example 3

Another example of data measured and calculated by the system described herein is in Table 3 below. Newborn Calf Serum commercially obtained was utilized as the target fluid. The variable pitch device with variable pitch platform as illustrated and disclosed in U.S. Pat. No. 7,736,494 were used as the gas-fluid contacting device. The following initial conditions were utilized; 900 ppmv ozone inlet concentration, 145 ml initial fluid volume, 1000 ml per minute gaseous flow rate, 189 ml per minute fluid flow rate counter current to the ozone/oxygen admixture flow. Incremental reductions in fluid volume are due to sampling of fluid through the fluid access port.

TABLE 3
NEWBORN CALF SERUM
MEASURED VARIABLES

| Elapsed-time | | | | Average Inlet Ozone | Average Exit Ozone |
|---|---|---|---|---|---|
| (5 minute | Fluid Volume | Gas Flow Rate | Fluid Flow Rate | Concentration | Concentration |
| intervals) | (milliliters) | (liters/minute) | (liters/minute) | (ppmv) | (ppmv) |
| 5 | 145 | 1.000 | 0.189 | 908.1 | 68.0 |
| 5 | 143 | 1.000 | 0.189 | 911.4 | 50.1 |
| 5 | 141 | 1.000 | 0.189 | 904.4 | 46.6 |
| 5 | 139 | 1.000 | 0.189 | 904.7 | 50.9 |

CALCULATED VARIABLES

| | Average | | | | |
|---|---|---|---|---|---|
| | Differential Ozone | Delivered- | Residual- | Ozone Absorbed | Absorbed-dose |
| Elapsed-time | Concentration | ozone | ozone | per Interval | of ozone |
| (minutes) | (ppmv) | (ug) | (ug) | (ug) | (ug) |
| 5 | 840.1 | 9.72E+03 | 7.28E+02 | 8.99E+03 | 8.99E+03 |
| 10 | 861.3 | 1.95E+04 | 1.26E+03 | 9.22E+03 | 1.82E+04 |
| 15 | 857.8 | 2.92E+04 | 1.76E+03 | 9.18E+03 | 2.74E+04 |
| 20 | 853.8 | 3.88E+04 | 2.31E+03 | 9.13E+03 | 3.65E+04 |

In one embodiment of the invention, a method is provided to treat acute ischemic brain stroke in a mammal. The method involves subjecting an amount of blood, blood fractionate or other biological fluid ex vivo to an amount of ozone delivered by an ozone delivery system. The method may also provide for the maintenance of the biological integrity of the treated fluid. Furthermore, the method describes treatment conditions for acute ischemic brain stroke at temperatures compatible with maintaining the biological integrity of biological fluids.

For blood products, the biological integrity of plasma may be measured by the functionality of its protein components either in whole plasma or after separation into plasma fractions. The biological integrity of red blood cell and platelet preparations may be determined by the methods and criteria known by those skilled in the art and are similar to those used in establishing the suitability of storage and handling protocols. In practical terms, the biological integrity of a biological fluid is a fluid that subsequent to the method of treating acute ischemic brain stroke described herein, has sufficiently maintained its functionality upon re-infusion into a mammalian patient.

Fluid-contacting surfaces including gas-fluid contacting devices constructed from ozone-inert material(s) may be treated with a human serum albumin (HSA) solution to prevent platelet adhesion, aggregation and other related platelet phenomena in the instances when a biological fluid to be treated contains platelets (i.e. whole blood, platelet concentrates). Generally, HSA solutions ranging between 1 and 10% may be employed. An HSA solution prepared in a biocompatible bacteriostatic buffer solution will be passaged throughout the gas-fluid contacting device. Subsequent to passage, the HSA solution will be drained from the device. The gas-fluid contacting device and all surfaces that are in contact with the biological fluid during the method described are now primed for use with platelet-containing biological fluids.

Another embodiment of the present invention comprises a method for treatment of acute ischemic brain stroke in a mammalian patient which involves removing blood directly from a subject and reinfusing it to the same patient in a continuous loop configuration. The blood may circulate through the loop, which includes a gas-fluid contacting device once, or multiple times, wherein a measured amount of ozone is delivered to the blood under conditions which may maintain the biological integrity of the blood. The treated blood is constantly re-infused directly back into the same patient.

Alternative applications of the methods of the present invention involve plasmapheresis wherein the patient's plasma is selectively removed while the balance of the blood cells is immediately returned to the patient. A measured amount of ozone is delivered to the isolated plasma under conditions which may maintain the biological integrity of the plasma. The treated plasma is subsequently reinfused into the subject.

In those aspects of the invention where the method of treatment involves a continuous loop approach, the volume of blood treated can range between can vary between 10 ml and the total estimated circulating blood volume of a mammalian patient being treated multiple times. Generally, the blood volume treated will range between 10 ml and 10000 ml and preferably range between 10 ml and 6000 ml.

In those aspects of the invention where the method of treatment involves a discontinuous approach, the volume of blood removed can range from 1 to 5000 ml, depending on patient size and blood volume. This discontinuous treatment approach may be performed once or multiple consecutive times during a single treatment.

The time required for an individual treatment through the use of a continuous loop format is based on a number of factors including the desired number of passes through the loop, volume of the fluid treated, the flow rate at which the fluid is circulating, the interface time required between the fluid and the amount of delivered-ozone, and the amount of the absorbed-dose of ozone required. The time for the treatment can range from 1 minute to 720 minutes and preferably range from 1 minute to 180 minutes.

The number and frequency of treatments can vary considerably based upon the clinical situation of a particular patient. Generally the number of treatments can range between an individual treatment and 200 treatments, to be provided on a daily, alternate day or other schedule based on the clinical evaluation of the patient and desired clinical outcomes. Upon completion of a number of treatments and evaluation by a health care practitioner, another course of treatments may be indicated.

The methods of the present invention are described for treatment of conditions attendant to ischemic brain stroke, comprising methods that employ ozone delivery devices that are constructed with all ozone-contacting surfaces being made or constructed of ozone-inert materials to assure accurate determination of the amount of ozone delivered to a fluid being treated, and to assure accurate determination of the amount of ozone absorbed by the fluid. The ozone delivery structures, and related methods of treating blood and other biological fluids with ozone, and the use of those fluids for therapeutic treatments as disclosed herein, may be varied from those described to adapt the structures and methods to specific applications. Therefore, reference to specific constructions and methods of use are by way of example and not by way of limitation.

What is claimed is:

1. A method for treating a mammalian subject suffering from, or believed to suffer from, acute ischemic brain stroke, comprising:
providing a biological fluid withdrawn from a mammalian subject;
processing said fluid in an ozone delivery system to deliver to said fluid a measured amount of ozone to effect absorption by the fluid of a quantifiable absorbed dose of ozone; and
reintroducing the treated fluid having a quantifiable absorbed dose of ozone to the mammalian subject to provide therapeutic treatment of acute ischemic brain stroke conditions and symptoms.

2. The method according to claim 1, wherein said processing of the fluid is carried out in an ozone delivery system all gas-contacting surfaces of which are constructed of ozone-inert materials.

3. The method according to claim 1, wherein said processing of the fluid is carried out in a discontinuous loop format.

4. The method according to claim 1, wherein said processing of the fluid is carried out in a continuous flow format.

5. The method according to claim 1, wherein said biological fluid is blood, a blood derivative or a blood fractionate.

6. The method according to claim 5, wherein said blood fractionate comprises separated cellular fractions or platelets.

7. The method according to claim 1, wherein said processing of said biological fluid is carried out in a manner to maintain the biological integrity of said fluid.

8. The method according to claim 1, wherein said therapeutic treatment further comprises eliciting a reduction in pro-inflammatory and/or an increase in anti-inflammatory cytokines released by immunomodulatory T cells.

9. The method according to claim 1, wherein said therapeutic treatment further comprises inducing sufficient leukocyte apoptosis, without excessive necrosis, to elicit clinical benefits.

10. The method according to claim 1, wherein the therapeutic treatment provides a reduction of edema associated with the ischemic penumbra, increased blood flow to the area surrounding an infarct, relaxation of the vascular endothelium or reduction of inflammation, and combinations of these effects.

11. The method according to claim 1, wherein the therapeutic treatment elicits increase in vasodilation, improvement in endothelial function, improvement in endothelial cellular repair or replacement or improvement in blood flow yielding enhanced oxygenation, and combinations of these effects.

12. A method of producing a therapeutic substance for the treatment of acute ischemic brain stroke and related symptoms or conditions, comprising:
providing a biological fluid;
delivering to the biological fluid a measured amount of ozone to produce a therapeutic substance having a quantifiable absorbed-dose of ozone which, upon administration to a subject suffering, or believed to be suffering, from acute ischemic brain stroke, effectively treats the symptoms and conditions related to the acute ischemic brain stroke.

13. The method according to claim 12, wherein said biological fluid is blood, a blood derivative or blood fractionate.

14. A medicament for the treatment of acute ischemic brain stroke, and the symptoms or conditions related thereto, comprising a biological fluid containing a quantifiable absorbed-dose of ozone to provide efficacious therapeutic effect to a subject suffering, or believed to be suffering, from acute ischemic brain stroke and the symptoms or conditions related thereto upon administration of the medicament to the subject.

15. The medicament according to claim 14, wherein said biological fluid is blood, a blood derivative or blood fractionate.

16. The medicament according to claim 15, wherein said blood fractionate is comprised of platelets.

17. The medicament according to claim 15, wherein said blood derivative is plasma.
